(19) United States
(12) Patent Application Publication
(10) Pub. No.: US 2016/0193336 A1
(43) Pub. Date: Jul. 7, 2016

(54) METHODS FOR USING NITRIC OXIDE IN A PLASMA STATE TO TREAT MEDICAL CONDITIONS AND DISEASES

(71) Applicant: ORIGIN, INC., Princeton, NJ (US)

(72) Inventors: Howard Nelson, Lawrenceville, NJ (US); Alexander Dolgopolsky, Richboro, PA (US); Michael D. Preston, Orangeburg, NY (US); Michael Pohl, Pelham, AL (US); Victor N. Vasilets, Chernogolovka (RU)

(21) Appl. No.: 14/911,397

(22) PCT Filed: Oct. 30, 2014
(86) PCT No.: PCT/US14/63075

§ 371 Date: Feb. 10, 2016

Related U.S. Application Data
(60) Provisional application No. 61/898,390, filed on Oct. 31, 2013.

(51) Int. Cl. A61K 41/00; A61K 9/00; A61K 33/00

(52) U.S. Cl. CPC A61K 41/0023; A61K 33/00; A61K 9/0014; A61K 9/007

(57) ABSTRACT

Methods for administering nitric oxide (NO) in a plasma state to a treatment site are disclosed. A discrete stream of matter is placed in a plasma state, in which the stream has, as part of its content, a desired concentration of NO. The discrete stream of matter is directed at a site of action to achieve a therapeutic result.

### CROSS-REFERENCE TO RELATED APPLICATION

The application is a non-provisional of, and claims priority to, pending U.S. provisional patent application Ser. No. 61/898,390, filed Oct. 31, 2013, titled “Methods for Using Nitric Oxide in a Plasma State to Treat Medical Conditions and Diseases,” the entirety of which application is incorporated by reference herein.

### FIELD OF THE DISCLOSURE

The disclosure relates generally to the field of medical treatment and more particularly to methods for effectively administering nitric oxide in the treatment of medical conditions and diseases.

### BACKGROUND OF THE DISCLOSURE

Nitric Oxide (NO) gas is a short-lived molecule normally found in a gaseous state both inside and outside the human body. NO is a signaling molecule known to have numerous regulatory, protective and therapeutic properties. Augmenting the body's natural generation of NO by either stimulating increased production of endogenous NO or introducing exogenously-produced NO into the body can improve the body's response to damage, pain, and invading organisms. However, it is difficult to deliver NO into living tissue and, in its gaseous state, NO does not penetrate through the dermis. To be clinically useful, NO must be present in the site of action in a sufficient quantity.

Prior methods for delivering NO for therapeutic purposes include the administration of chemical compounds which release NO chemically into the body. Other methods employ NO pathway agonists and NO antagonists. Still other methods employ high pressure NO gas and sprays. Yet another method involves surrounding a body with sealed vacuum containers into which gaseous NO is introduced. Attempts have also been made to force pressurized nitric oxide through tissue and skin. For various reasons, these methods have yielded limited results. For example, gaseous NO is highly reactive, has low diffusion constant and has extremely short life-time in tissue media.

Another method that has failed to achieve clinical success involves the administration of molecular donors, which has been demonstrated to be problematic because the control of the release of the payload cannot be modulated, nor can the penetration/saturation of the donors be reliably modulated.

There are several solutions that target specific clinical outcomes involving NO. Sildenafil citrate (sold under the brand name VIAGRA), for example, interferes with the down regulation of NO in erectile dysfunction syndrome. Etanercept (sold under the brand name ENBRIL), for example, uses an anti-TNF alpha antibody to do what NO would do in inflammatory diseases of the joint. Most solutions involve affecting the NO pathways, due to the difficulty in stimulating production of NO directly at the site of action. Because of the lack of site specificity of these NO pathway pharmacologics, negative side effects can be serious.

### SUMMARY

In view of the forgoing, it would be advantageous to provide a method for administering NO at a site of action in a manner that facilitates therapeutic benefits.

In accordance with the present disclosure, therapeutic methods of administering NO to achieve a therapeutic benefit are disclosed. In some embodiment, the methods include employing the exogenous production and application of NO by high temperature plasma conversion of air. In other embodiments, the NO is applied to a treatment site to facilitate repair and growth of living tissue in animals, humans and plants.

This method of the present disclosure operates to selectively apply NO to a treatment site for the beneficial effects evident with increased NO levels associated with the cellular and tissue environment. The methods more particularly include employing an apparatus capable of producing matter in a plasma state having a desired composition including NO. The apparatus may be used to apply a desired level of NO, via matter in a plasma state, to a treatment site.

### BRIEF DESCRIPTION OF THE DRAWINGS

FIG. 1 illustrates a first exemplary device for producing NO according to the disclosure;

FIG. 2 illustrates a second exemplary device for producing NO according to the disclosure;

FIG. 3 illustrates a third exemplary device for producing NO according to the disclosure; and

FIG. 4 is a flow diagram illustrating a first exemplary method in accordance with an embodiment of the present invention.

### DETAILED DESCRIPTION

The present invention will now be described more fully hereinafter with reference to the accompanying drawings, in which preferred embodiments of the invention are shown. The invention, however, may be embodied in many different forms and should not be construed as being limited to the embodiments set forth herein. Rather, these embodiments are provided so that this disclosure will be thorough and complete, and will fully convey the scope of the invention to those skilled in the art. In the drawings, like numbers refer to like elements throughout.

In accordance with the present disclosure, a method and apparatus are presented for creating a discrete stream of matter in a plasma state, where the stream has as part of its content NO, and administering such stream to an organism to obtain a therapeutic result. In some embodiments, NO application at the surface level (i.e., directed at the skin or open wound) is believed to stimulate the body's own production of NO such that therapeutic effects can be obtained at and around the indication site. Alternatively, the disclosed methods may exploit the fact that NO in the plasma state is of sufficiently high energy and velocity that it can penetrate through and around cellular membranes. In some cases the NO may pass through biofilms and the stratum corneum to produce therapeutic results in the associated tissue.

Matter in a plasma state that contains NO can be created via several methods. Atmosphere contains nitrogen and oxygen, and thus, sufficient energy in the correct geometry can produce nitric oxide from the gaseous mixture. Energy can be added to transition the gaseous N2 and O2 into the plasma state. In one non-limiting, exemplary embodiment, pre-formed gaseous in N2-O2 mixture can be created and passed through a plasma energy arc that transfers sufficient energy to production of NO in a plasma state.

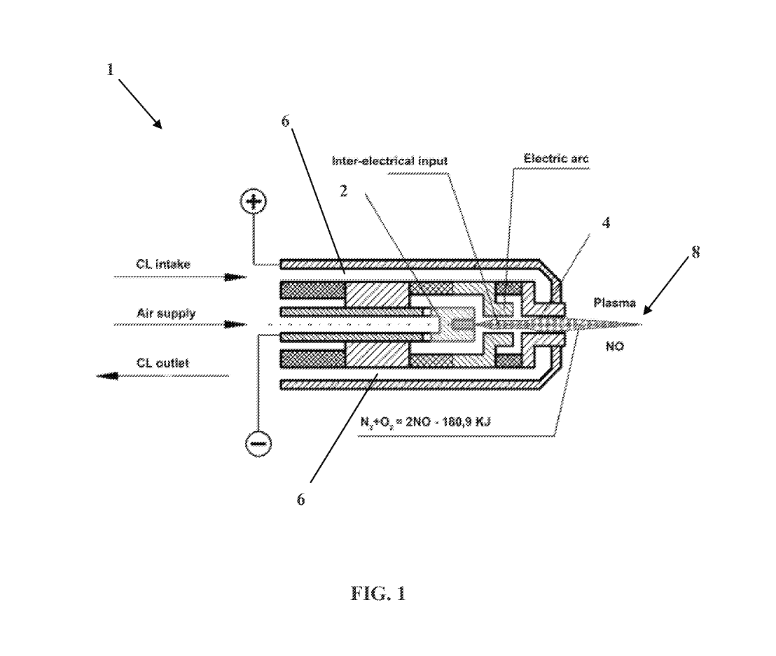

FIG. 1 shows an exemplary generator portion of a NO production device 1 for use in carrying out one or more of the disclosed methods. As can be seen, air is introduced at a first end of the device 1, and is channeled between a pair of electrodes, namely a cathode 2 and an anode 4 which are insulated from each other. A stationary DC arc discharge is generated and maintained between the electrodes 2, 4. A NO-containing gas flow is formed from the air in the area between the electrodes 2, 4 under the effect of the arc discharge, and is withdrawn through a cooled channel (cooled by a coolant loop 6), enabling NO to be fixed in the flow 8. The temperature of the flow and the NO content can be brought to desired values for providing a therapeutic benefit to a treatment site. Further details regarding the illustrated exemplary NO production device 1 can be found, for example, in U.S. Pat. No. 7,498,000 to Pekshev, the entirety of which is incorporated herein by reference.

Classical thermodynamics confirmed by compositional analysis of the thermodynamical equilibrium of air in a plasma state shows that at a temperature lower than 2000° C. the concentration of NO in the gas does not exceed 1%. Increasing the plasma temperature increases the NO concentration up to its maximum (˜5%) at a temperature of 3500-4000° C. Slightly less than 4000° C. is the temperature of the electrical discharge in the plasma arc of the illustrated device 1. Plasma-chemical reactions, which lead to the formation NO, can be expressed by the following chemical formula:

N₂+O₂→2NO−180.9 kilo-Joules (kJ)

Life-time of the NO molecule at high temperatures is comparable to the time of its synthesis because of fast reaction of recombination (2NO+O₂=2NO₂)+. To prevent recombination and keep therapeutic concentration of NO for medical applications, it is desirable to accomplish rapid cooling of the reaction mixture, i.e., quenching. Quenching of the NO occurs with braking of the escaping flow in surrounding cold air (i.e., from coolant loop 6). The illustrated device 1 enables a direct current plasma arc to be created using ambient air at atmospheric pressure. The device 1 produces a stream of hot air 8 with a composition of plasma species that contains medically significant amount of NO, which in one exemplary embodiment is about 2,500 parts per million (ppm) NO.

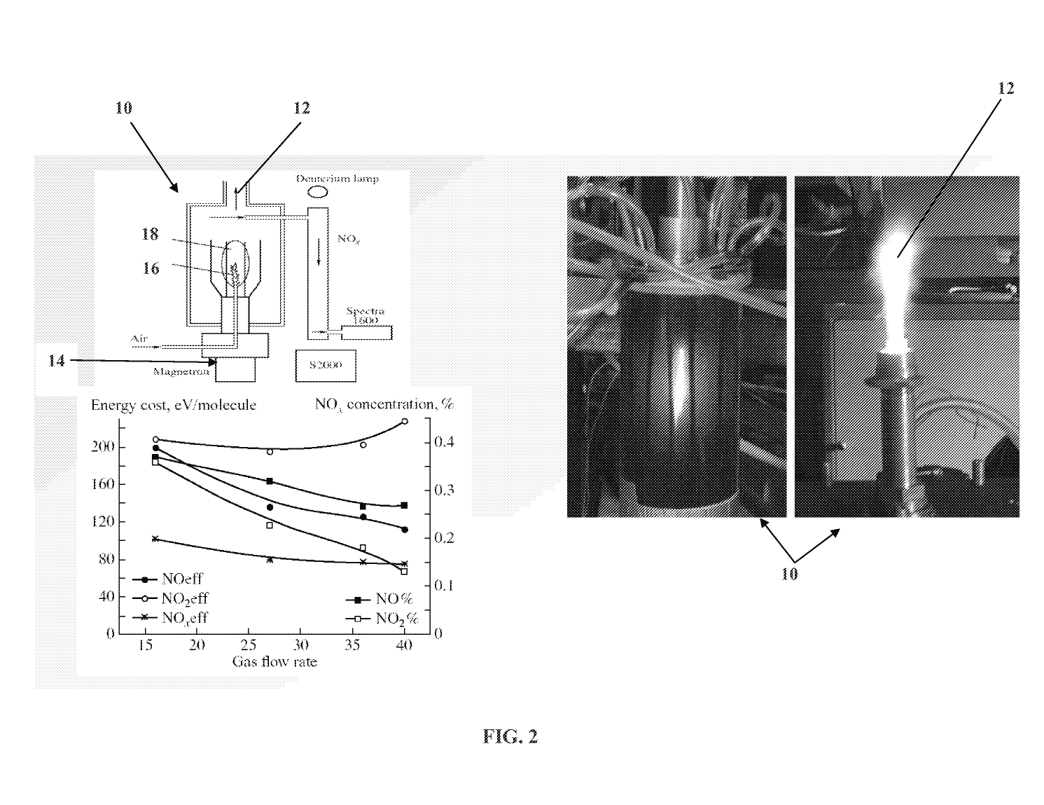

The device 1 shown in FIG. 1 is not exclusive, and alternative sources of plasma-generated NO may also be used to carrying out one or more of the disclosed methods. FIG. 2 shows such an alternative device 10 for production of NO-containing matter in a plasma state 12 for use in carrying out one or more of the disclosed methods. This device 10 employs microwave discharge technology for producing matter in a plasma state 12 having a desired composition (i.e., about 2,000 ppm of NO). The illustrated device 10 includes a magnetron 14 having a power of P<1 kilowatt (kW) and a frequency of 2.45 gigahertz (GHz). Air is passed by the magnetron 14 and directed to a core portion 16 of the torch body 18, where a stream of matter in a plasma state 12 is generated and output for application to a targeted treatment site.

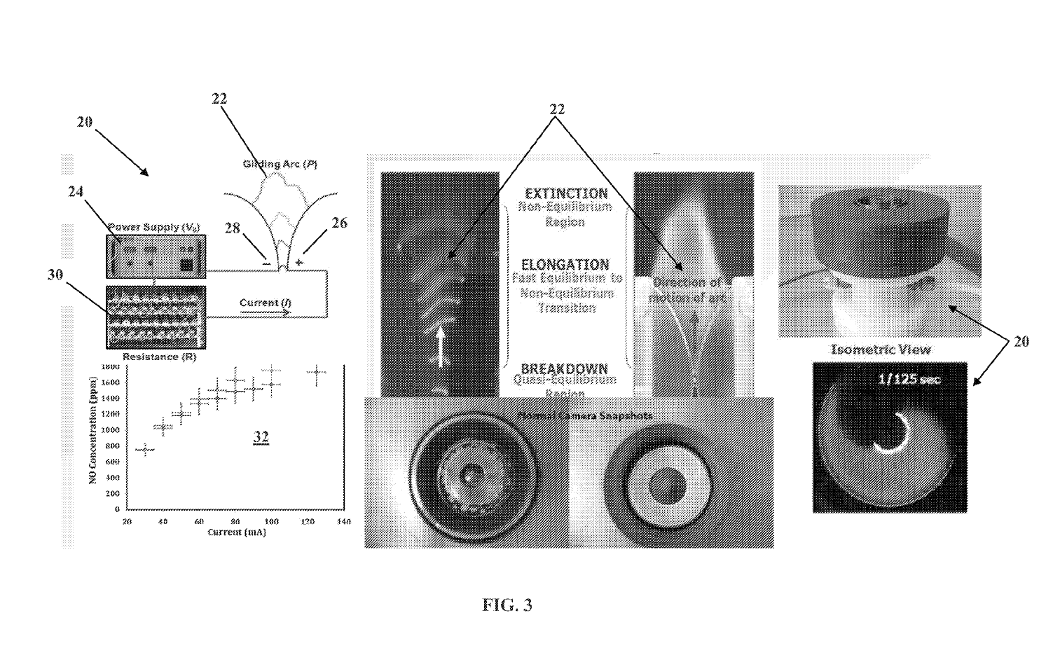

FIG. 3 shows yet another device 20 for production of NO-containing matter in a plasma state for use in carrying out one or more of the disclosed methods. The illustrated device 20 employs magnetically stabilized gliding arc discharge technology for producing matter in a plasma state 22 having a desired composition (again, about 2,000 ppm of NO).

A gliding arc is operated in air at atmospheric pressure, but at moderate power levels (typically between 50 and 300 Watts). A power source 24 and anode/cathode 26, 28 is employed, and current is restricted using an external ballast resistor 30. This heats the discharge (i.e., the plasma jet 22) to moderate temperatures (2000-3000 degrees Kelvin), while preserving non-equilibrium nature of the discharge (Te>Tg). As such, higher concentrations of NO (e.g., 1600-1800 ppm) can be obtained at lower power input. A graph 32 shows the relationship between NO concentration (ppm) of the matter in a plasma state 22 vs. discharge current (mA).

As will be appreciated, NO in a plasma state can be used for a variety of purposes. For example, NO in the plasma state can be used as an antimicrobial agent. In addition, NO in the plasma state can be used to facilitate hair-growth, as an anti-wrinkle agent, to reduce inflammation, or to facilitate vasodilation. NO in the plasma state further can be employed to alleviate pain associated with osteoarthritis and Rheumatoid Arthritis. It can also be effective in combating Gram Positive microorganisms, Gram Negative microorganisms, Fungi (including onychomycosis) and viruses. It is also therapeutic in treating osteoporosis, collagen formation, stem cell signaling, satellite cell differentiation, wound-healing, wound-management, reduction in scar tissue, remediation of activity related injury, and acne. NO in a plasma state can also aid in nerve regeneration, can inhibit cancer cell proliferation, can promote apoptosis, can stimulate endogenous nitric oxide production, and can stimulate iNOS pathways.

In practice, the NO in a plasma state can be applied directly to or adjacent to living tissue in order to produce the desired effect. It can effectively function to maintain homeostasis in the cardiovascular and respiratory systems. NO, as a signaling molecule, can cause vasodilation which promotes blood vessel flexibility, eases blood pressure, cleans the blood, reverses atherosclerosis and effectively prevents cardiovascular diseases and aids in its recovery. Another important function of NO is slowing down atherosclerotic plaque deposition on vascular walls. NO also plays an active defense role in the immune system. It is a strong antioxidant, and can suppress bacterial infections, viruses and parasitic attacks. It can even deter some types of cancer cell growth. In patients with moderate to severe diabetes, NO can prevent many common and serious complications. NO can also significantly reduce the pain associated with joint swelling in arthritis. NO can effectively decrease the risk of cancer, diabetes, myocardial infarction and stroke.

In the nervous and endocrine systems, NO can induce normal functioning of various body organs. NO can permeate freely through the cell membrane for biological signaling, adjust cellular activities and lead every organ to complete its function properly, including the lungs, liver, kidneys, stomach, heart, brain and genitals. NO can increase blood flow to the genital organs to maintain normal sexual function. The brain transmits signals via its surrounding nerves to the perineal region to provide it with sufficient NO to cause vascular dilation, increasing blood flow to enhance erectile function. Under some conditions, weak erections are the results of insufficient NO production by nerve endings.

NO can also slow the aging process and improve memory. The NO molecules produced by the immune system are not only capable of destroying invading microorganisms, but also help activate and nourish brain cells, significantly slowing aging and improving memory.

### Exemplary Indications

A non-limiting listing of exemplary indications for which the disclosed NO-containing matter in a plasma state may find beneficial use as a treatment includes:

Skin Infections and Wound-Healing

Skin Infections
In chronic wounds
   Gram-positive
   Gram-negative
In acute wounds
   Gram-positive
   Gram-negative
In sub-acute wounds
   Gram-positive
   Gram-negative

Fungal Infections
Tinea pedis
Onychomycosis
   Toes
   Fingers

Wound-Healing
Chronic
   Diabetic foot ulcers
   Decubitus ulcers
Acute
Sub-acute
Burns
   First degree
   Second degree
   Third degree

### Pain and Inflammation

Osteo-Arthritis
Knee
Hand
Ankle
Shoulder
Elbow
Toe
Spine and neck
Hip
Finger

Rheumatoid Arthritis
Knee
Hand
Ankle
Shoulder
Elbow
Toe
Spine and neck
Hip
Finger

An exemplary baseline composition of matter in a plasma state is shown in Table 1 below. This baseline composition of matter can be employed with any of the following Exemplary Treatment Schemes, which will be described in more detail later. It will be appreciated, however, that this composition itemization is merely exemplary, and that other compositions can also be used to beneficial effect.

TABLE 1
BASELINE PLASMA COMPOSITION

| | | |
|---|---|---|
| | Minimum | Maximum |
| Parameter | Value | Value |

| | | | | |
|---|---|---|---|---|
| Nitric Oxide (NO) | 200 | ppm | 1000 | ppm |
| NO₂ | 0 | ppm | 25 | ppm |
| N₂ | 75 | vol % | 78 | vol % |
| O₂ | 18 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

An exemplary baseline treatment scheme is shown in Table 2 below. This baseline treatment scheme can be employed with any of the following Exemplary Treatment Schemes. It will be appreciated that this baseline scheme may be adjusted, as will be described in relation to a number of Examples to follow, to provide a desired treatment plan for an affected area and in response to a particular indication.

As shown in Table 2, the treatment variables include “distance from exit to site,” “time of application,” “number of treatments,” “length of time between treatments,” “temperature of plasma stream at contact with site,” and “velocity of plasma stream at contact with treatment site.”

“Distance from exit to site” will be understood to be the standoff distance, in centimeters, from the outlet of the plasma device (e.g., device 1, 10, 20) to the treatment site. “Time of application” will be understood to be the amount of time, in seconds, that the NO-containing matter in a plasma state will be directed from the plasma device onto the treatment site, per square centimeter of site area. Thus, the time of application will depend upon the size of the area being treated. “Number of treatments” will be understood to be the discrete number of treatments to be applied at the site. “Length of time between treatments” will be understood to be the amount of time elapsed between applications of the NO-containing matter in a plasma state at the treatment site. “Temperature of plasma stream at contact with treatment site” will be understood to be the temperature of the NO-containing matter in a plasma state, in degrees Celsius, at the treatment site. “Velocity of plasma stream at contact with treatment site” will be understood to be the speed of the NO-containing matter in a plasma state, in meters per second, at the treatment site. Minimum and maximum values are provided for each, recognizing that individual treatment specifications for particular indications will vary within the indicated ranges.

TABLE 2
BASELINE TREATMENT SCHEME

| | Minimum | Maximum |
|---|---|---|
| Parameter | Value | Value |
| Distance from exit to site (cm) | 1 | 25 |
| Time of Application (sec/cm²) | 5 | 45 |
| Number of treatments | 1 | 24 |
| Length of time between treatments (hours) | 3 | 168 |
| Temperature of plasma stream at contact with | 10 | 60 |
| treatment site ° C. | | |
| Velocity of Plasma Stream at contact with | 0.5 | 200 |
| treatment site (m/sec) | | |

A series of Exemplary Treatment Schemes will now be discussed in relation to various indications. Except where indicated otherwise, these treatment schemes assume use of matter in a plasma state having the compositions identified in Table 1.

### Example 1

### Treatment Scheme

### Gram Positive Bacteria

See Table 1B below, for partial list of gram positive pathogens. See Table 1C, below, for partial list of conditions that present with pathogens from Table 1B.

The minimum treatment values and maximum treatment values are identified below are based on severity of the gram positive bacterial infection. Severity of the infection is determined by the surface area, depth, colony count and symptoms. Application of therapy increases in intensity, duration and frequency as the severity increases. Minimum treatment parameters define the requirements for the initiation of the decolonization process.

### Example 1

TABLE 1A

| | Minimum | Maximum |
|---|---|---|
| Parameter | Value | Value |
| Distance from exit to site (cm) | 1 | 25 |
| Time of Application (sec/cm²) | 5 | 45 |
| Number of treatments | 1 | 24 |
| Length of time between treatments (hours) | 3 | 168 |
| Temperature of plasma stream at contact | 10 | 60 |
| with Site ° C. | | |
| Velocity of Plasma Stream at contact | 0.5 | 200 |
| with Site (m/sec) | | |

### Example 1

TABLE 1B
Pathogens Susceptible to Plasma/NO therapy

MRSA
MDSA
Staphylococcus Aureus
Streptococcus A
Streptococcus B
C. Difficile
Streptococcus Mutans
Myco Bacterium Tuberculosis
Bacillus subtilis
Streptococcus Pneumoniae
Vancomycin Resistant Enterococcus Faecium

### Example 1

TABLE 1C
Conditions that Present with Pathogens in Table 1B

Venous Ulcers
Pressure Ulcers
Decubitus Ulcers
Sickle Cell Ulcerations
Pyodermas
Ulcerated Lesions
Vasculitis
Diabetic Foot Ulcers
Folliculitis
Cellulitis
Myositis
Infections from Animal Bites
Surgical Site Infections
Catheterizations
Carbuncles
Furuncles
Abscesses
Erysipeloid
Erysipelas
Keratolysis
Dermatitis
Skin Tuberculosis
Impetigo
Actinomytosis
Leishmaniasis
Herpes Simplex
Herpetic Neuralgia
Skin Flaps
Skin Grafts
Burns
Traumatic Wounds
Complicated SSTI

### Example 2

### Treatment Scheme

### Gram Negative Bacteria

See Table 2B for partial of gram negative pathogens. See Table 2C for partial list of conditions that present with pathogens from Table 2B.

The minimum treatment values and maximum treatment values are based on severity of the gram negative bacterial infection. Severity of the infection is determined by the surface area, depth, colony count and symptoms. Application of therapy increases in intensity, duration and frequency as the severity increases. Minimum treatment parameters define the requirements for the initiation of the decolonization process. Gram negative bacteria are more difficult to kill than gram positive, so longer treatments are required to decolonize.

### Example 2

TABLE 2A

| | Minimum | Maximum |
|---|---|---|
| Parameter | Value | Value |
| Distance from exit to site (cm) | 1 | 25 |
| Time of Application (sec/cm²) | 10 | 90 |
| Number of treatments | 1 | 24 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

### Example 2

TABLE 2B
Gram Negative Pathogens

Escherichia coli
Salmonella
Klebsiella pneumoniae
Serratia marcescens
Enterobacter aerogenes
Stenotrophomonas maltopilia
Pseudomonas aeruginosa
Acinetobacter baumannii
Proteus Vulgaris
Pantoea agglomerans

### Example 2

TABLE 2C
Conditions That Present with Pathogens in Table 2B

Venous Ulcers
Pressure Ulcers
Decubitus Ulcers
Sickle Cell Ulcerations
Pyodermas
Ulcerated Lesions
Vasculitis
Diabetic Foot Ulcers
Folliculitis
Cellulitis
Myocitis
Infections from Animal Bites
Surgical Site Infections
Catheterizations
Carbuncles
Furuncles
Abscesses
Erysipeloid
Erysipelas
Keratolysis
Dermatitis
Skin Tuberculosis
Impetigo
Actinomytosis
Leishmaniasis
Herpes Simplex
Herpetic Neuralgia
Skin Flaps
Skin Grafts
Burns
Traumatic Wounds
Complicated SSTI

### Example 3

### Treatment Scheme

### Wounds—Pressure Ulcers

Severity Classification subject to Table 3B. Clinical Presentation subject to Table 3C.

The minimum treatment values and maximum treatment values are based on severity of pressure ulcer wound. Severity of the infection is determined by the surface area, depth, and symptoms. Application of therapy increases in intensity, duration and frequency as the severity increases. Minimum treatment parameters define the requirements for the initiation of the wound care management process.

### Example 3

TABLE 3A

| | Minimum | Maximum |
|---|---|---|
| Parameter | Value | Value |
| Distance from exit to site (cm) | 1 | 25 |
| Time of Application (sec/cm²) | 1 | 45 |
| Number of treatments | 1 | 200 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

### Example 3

TABLE 3B
Deep Tissue Wounds (Pressure Ulcer Classification)

Stage I - Intact skin with non blanchable redness usually over a
bone
Stage II - Partial thickness loss of dermis with open ulcer
Stage III - Full thikness tissue loss sub-cutaneous fat may be
exposed but not bone, tendon or muscle
Stage IV - Full thickness tissue loss with exposed bone, tendon or
muscle
Unstageable - Full thickness tissue loss in which the base of the
ulcer is covered by slough, until it is removed to expose the base
of the wound the true depth and therefore stage cannot be
determined.

### Example 3

TABLE C
Other Classifications of Wounds

Surgical
Traumatic
Chronic
Acute
Sub-dermal
Dermal

### Example 4

### Treatment Scheme

### Wounds—Neuropathic Ulcers

Severity Classification is subject to Table 4B. Clinical Presentation is subject to Table 4C.

The minimum treatment values and maximum treatment values are based on severity of neuropathic ulcer wound. Severity of the wound is determined by the surface area, depth, and symptoms. Application of therapy increases in intensity, duration and frequency as the severity increases. Minimum treatment parameters define the requirements for the initiation of the wound care management process.

### Example 4

TABLE 4A

| | Minimum | Maximum |
|---|---|---|
| Parameter | Value | Value |
| Distance from exit to site (cm) | 1 | 25 |
| Time of Application (sec/cm²) | 5 | 90 |
| Number of treatments | 1 | 200 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

### Example 4

TABLE 4B
Severity Classification

Grade 1 - Superficial ulcer
Grade 2 - Penetration into tendon or joint capsule
Grade 3 - Involvement of deeper tissues
Grade 4 - Gangrene of the forefoot
Grade 5 - Gangrene involving more than two-
thirds of the foot

### Example 4

TABLE 4C
Other Classifications of Wounds

Surgical
Traumatic
Chronic
Acute
Sub-dermal
Dermal

### Example 5

### Treatment Scheme

### Wounds—Venous Ulcers

Severity Classification is subject to Table 5B. Clinical Presentation is subject to Table 5C.

The minimum treatment values and maximum treatment values are based on severity of pressure venous wound. Severity of the wound is determined by the surface area, depth, and symptoms. Application of therapy increases in intensity, duration and frequency as the severity increases. Minimum treatment parameters define the requirements for the initiation of the wound care management process. Treatment includes a border around the wound site of up to 4 cm due to circulatory issues.

### Example 5

TABLE 5A

| | Minimum | Maximum |
|---|---|---|
| Parameter | Value | Value |
| Distance from exit to site (cm) | 1 | 25 |
| Time of Application (sec/cm²) | 5 | 45 |
| Number of treatments | 1 | 24 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

### Example 5

TABLE 5B
Severity Classification

Grade 1 - Superficial ulcer
Grade 2 - Penetration into tendon or joint capsule
Grade 3 - Involvement of deeper tissues
Grade 4 - Gangrene of the forefoot
Grade 5 - Gangrene involving more than two-thirds of the foot

### Example 5

TABLE 5C
Other Classifications of Wounds

Surgical
Traumatic
Chronic
Acute
Sub-dermal
Dermal

### Example 6

### Treatment Scheme

### Wounds—Burns

Severity Classification is subject to Table 6B.

The minimum treatment values and maximum treatment values are based on severity of the burn. Severity of the burn is determined by the surface area, depth, and symptoms. Application of therapy increases in intensity, duration and frequency as the severity increases. Distance from the burn site dependent on patient's pain threshold. Minimum treatment parameters define the requirements for the initiation of the burn care management process.

### Example 6

TABLE 6A

| | Minimum | Maximum |
|---|---|---|
| Parameter | Value | Value |
| Distance from exit to site (cm) | 10 | 30 |
| Time of Application (sec/cm²) | 10 | 90 |
| Number of treatments | 1 | 200 |
| Length of time between treatments (hours) | 1 | 168 |
| Temperature of plasma stream at contact with | 10 | 50 |
| Site ° C. | | |
| Velocity of Plasma Stream at contact with Site | 0.5 | 200 |
| (m/sec) | | |

### Example 6

TABLE 6B
Severity Classification

Stage I Superficial
Stage II Superficial partial thickness skin loss
Stage III Deep partial thickness skin loss
Stage IV Full thickness dermal
Stage V Subdermal extending into muscle

### Example 7

### Treatment Scheme

### Osteoarthritis—Small Joints

See Table 7B for list of locations on body where the Small Joint treatment protocol applies.

The minimum treatment values and maximum treatment values are based on severity of inflammation, mobility and pain. Severity of the arthritis is determined by the level of inflammation, mobility and pain symptoms. Application of therapy increases in intensity, duration and frequency as the severity increases. Minimum treatment parameters define the requirements for the initiation of the osteoarthritis care management process. Treatment includes a border around the wound site of up to 1 cm due to circulatory issues.

### Example 7

TABLE 7A

| | Minimum | Maximum |
|---|---|---|
| Parameter | Value | Value |
| Distance from exit to site (cm) | 5 | 25 |
| Time of Application (sec/cm²) | 10 | 45 |
| Number of treatments | 3 | 40 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

### Example 7

TABLE 7B
Body Locations

Fingers
Toes
Hand
Wrist
Elbows
Ankles

### Example 8

### Treatment Scheme

### Osteoarthritis—Large Joints

See Table 8B for list of locations on body where the Large Joint treatment protocol applies.

The minimum treatment values and maximum treatment values are based on severity of inflammation, mobility and pain. Length of time is different from small joint due to the depth of the joint beneath the surface of the skin and the amount of surrounding soft tissue. Severity of the osteoarthritis is determined by the level of inflammation, mobility and pain symptoms. Application of therapy increases in intensity, duration and frequency as the severity increases. Minimum treatment parameters define the requirements for the initiation of the osteoarthritis care management process. Treatment includes a border around the wound site of up to 1 cm due to circulatory issues.

### Example 8

TABLE 8A

| | Minimum | Maximum |
|---|---|---|
| Parameter | Value | Value |
| Distance from exit to site (cm²) | 1 | 25 |
| Time of Application (sec/cm²) | 15 | 120 |
| Number of treatments | 3 | 50 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

### Example 8

TABLE 8B
Body Locations

Knee
Hip
Shoulder
Spine
Neck

### Example 9

### Treatment Scheme

### Rheumatoid Arthritis—Small Joints

See Table 9B for list of locations on body where the Small Joint treatment protocol applies.

The minimum treatment values and maximum treatment values are based on severity of inflammation, mobility and pain. Severity of the rheumatoid arthritis is determined by the level of inflammation, mobility and pain symptoms. Application of therapy increases in intensity, duration and frequency as the severity increases. Minimum treatment parameters define the requirements for the initiation of the rheumatoid arthritis care management process. Treatment includes a border around the wound site of up to 3 cm due to circulatory issues.

### Example 9

TABLE 9A

| | Minimum | Maximum |
|---|---|---|
| Parameter | Value | Value |
| Distance from exit to site (cm) | 3 | 25 |
| Time of Application (sec/cm²) | 30 | 90 |
| Number of treatments | 3 | 40 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

### Example 9

TABLE 9B
Body Locations

Fingers
Toes
Hand
Wrist
Elbows
Ankles

### Example 10

### Treatment Scheme

### Rheumatoid Arthritis—Large Joints

See Table 10B for list of locations on body where the Large Joint treatment protocol applies

The minimum treatment values and maximum treatment values are based on severity of inflammation, mobility and pain. Length of time is different from small joint due to the depth of the joint beneath the surface of the skin and the amount of surrounding soft tissue. Severity of the rheumatoid arthritis is determined by the level of inflammation, mobility and pain symptoms. Application of therapy increases in intensity, duration and frequency as the severity increases. Minimum treatment parameters define the requirements for the initiation of the rheumatoid arthritis care management process. Treatment includes a border around the wound site of up to 1 cm due to circulatory issues.

### Example 10

TABLE 10A

| | Minimum | Maximum |
|---|---|---|
| Parameter | Value | Value |
| Distance from exit to site (cm) | 1 | 25 |
| Time of Application (sec/cm²) | 20 | 120 |
| Number of treatments | 3 | 60 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

### Example 10

TABLE 10B
Body Locations

Knee
Hip
Shoulder
Spine
Neck

### Example 11

### Treatment Scheme

### Hair Follicle Stimulation

An exemplary composition of matter in a plasma state for use in hair follicle stimulation is shown in Table 11A below. It will be appreciated that this composition itemization is merely exemplary, and that other compositions can also be used to beneficial effect. Minimum treatment values and maximum treatment values in Tables 11A and 11B are based on skin type and age. Minimum treatment parameters define the requirements for initiation of the follicle stimulation process.

### Example 11

TABLE 11A
PLASMA COMPOSITION

| | Minimum | | Maximum | |
|---|---|---|---|---|
| Parameter | Value | | Value | |
| Nitric Oxide (NO) | 200 | ppm | 800 | ppm |
| NO₂ | 0 | ppm | 25 | ppm |
| N₂ | 75 | vol % | 95 | vol % |
| O₂ | 3 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

### Example 11

TABLE 11B

| Parameter | Minimum Value | Maximum Value |
|---|---|---|
| Distance from exit to site (cm) | 6 | 25 |
| Time of Application (sec/cm²) | 10 | 30 |
| Number of treatments | 10 | 100 |
| Length of time between treatments | 12 | 72 |
| (hours) | | |
| Temperature of plasma stream at | 10 | 60 |
| contact with Site ° C. | | |
| Velocity of Plasma Stream at | 0.5 | 200 |
| contact with Site (m/sec) | | |

### Example 12

### Treatment Scheme

### Post Insult Keloidosis

The disclosed use of NO in this Example is for treating keloidosis (scar tissue) that has occurred after an insult for the reduction of keloided mass in the insult site. An exemplary composition of matter in a plasma state for use in treating post insult keloidosis is shown in Table 12A below. It will be appreciated that this composition itemization is merely exemplary, and that other compositions can also be used to beneficial effect. Minimum treatment values and maximum treatment values in Tables 12A and 12B are based on depth and breadth of the keloided tissue. Application of therapy increases in intensity, duration and frequency as the severity increases. Minimum treatment parameters define the requirements for the initiation of the conversion of keloided tissue process.

### Example 12

TABLE 12A
PLASMA COMPOSITION

| Parameter | Minimum Value | | Maximum Value | |
|---|---|---|---|---|
| Nitric Oxide (NO) | 200 | ppm | 800 | ppm |
| NO₂ | 0 | ppm | 25 | ppm |
| N₂ | 75 | vol % | 95 | vol % |
| O₂ | 3 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 0 | ppm | 500 | ppm |
| H2 | 0 | ppm | 10000 | ppm |

### Example 12

TABLE 12B

| | Minimum | Maximum |
|---|---|---|
| Parameter | Value | Value |
| Distance from exit to site (cm) | 6 | 25 |
| Time of Application (sec/cm²) | 5 | 30 |
| Number of treatments | 4 | 24 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

### Example 13

### Treatment Scheme

### Pre Insult Keloidosis

This disclosed use of NO in this Example is for reducing treating keloidosis (scar tissue) that is anticipated to occur before an insult (such as a surgical incision) for the reduction of keloided mass in the subsequent insult site. An exemplary composition of matter in a plasma state for use in treating pre insult keloidosis is shown in Table 13A below. It will be appreciated that this composition itemization is merely exemplary, and that other compositions can also be used to beneficial effect. Minimum treatment values and maximum treatment values in Tables 13A and 13B are based on depth and breadth of the keloided tissue. Application of therapy increases in intensity, duration and frequency as the severity increases. Minimum treatment parameters define the requirements for the initiation of the conversion of keloided tissue process.

### Example 13

TABLE 13A
PLASMA COMPOSITION

| | Minimum | | Maximum | |
|---|---|---|---|---|
| Parameter | Value | | Value | |
| Nitric Oxide (NO) | 200 | ppm | 800 | ppm |
| NO₂ | 0 | ppm | 25 | ppm |
| N₂ | 75 | vol % | 95 | vol % |
| O₂ | 3 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

### Example 13

TABLE 13B

| | Minimum | Maximum |
|---|---|---|
| Parameter | Value | Value |
| Distance from exit to site (cm) | 6 | 25 |
| Time of Application (sec/cm²) | 10 | 30 |
| Number of treatments | 1 | 12 |
| Length of time between treatments (hours) | 3 | 72 |
| Temperature of plasma stream at contact with | 10 | 60 |
| Site ° C. | | |
| Velocity of Plasma Stream at contact with Site | 0.5 | 200 |
| (m/sec) | | |

### Example 14

### Treatment Scheme

### Asthma

This disclosed use of NO in this Example is for inhalation of NO to treat the signs and symptoms of asthma either as rescue therapy or maintenance therapy against an attack. An exemplary composition of matter in a plasma state for use in treating asthma is shown in Table 14A below. It will be appreciated that this composition itemization is merely exemplary, and that other compositions can also be used to beneficial effect. Minimum treatment values and maximum treatment values in Tables 14A and 14B are based on severity of asthmatic episode. Severity of the asthma is determined by the symptoms. Application of therapy increases in intensity, duration and frequency as the severity increases. Minimum treatment parameters define the requirements for the initiation of the asthma management process.

### Example 14

TABLE 14A
PLASMA COMPOSITION

| | Minimum | | Maximum | |
|---|---|---|---|---|
| Parameter | Value | | Value | |
| Nitric Oxide (NO) | 50 | ppm | 800 | ppm |
| NO₂ | 0 | ppm | 25 | ppm |
| N₂ | 75 | vol % | 78 | vol % |
| O₂ | 18 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

### Example 14

TABLE 14B

| | Minimum | Maximum |
|---|---|---|
| Parameter | Value | Value |
| Distance from exit to site (cm) | 8.5 | 25 |
| Applicant (inhalation) breaths | 2 | 45 |
| Number of treatments | 1 | 42 |
| Length of time between treatments (hours) | 3 | 48 |
| Temperature of plasma stream at contact with | 10 | 40 |
| Site ° C. | | |
| Velocity of Plasma Stream at contact with Site | 0.5 | 200 |
| (m/sec) | | |

### Example 15

### Treatment Scheme

### Respiratory Infections

This disclosed use of NO in this Example is for inhalation of NO to treat the bacterial lode of gram positive or gram negative infections of the lungs and sinuses and other airways. An exemplary composition of matter in a plasma state for use in treating such infections is shown in Table 15A below. It will be appreciated that this composition itemization is merely exemplary, and that other compositions can also be used to beneficial effect. Minimum treatment values and maximum treatment values in Tables 15A and 15B are based on severity of infection. Severity of the infection is determined by the symptoms. Application of therapy increases in intensity, duration and frequency as the severity increases.

Exemplary pathogens are included in Table 15C, while exemplary conditions are included in Table 15D.

### Example 15

TABLE 15A
PLASMA COMPOSITION

| | Minimum | | Maximum | |
|---|---|---|---|---|
| Parameter | Value | | Value | |
| Nitric Oxide (NO) | 50 | ppm | 800 | ppm |
| NO₂ | 0 | ppm | 25 | ppm |
| N₂ | 75 | vol % | 78 | vol % |
| O₂ | 18 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

### Example 15

TABLE 15B

| | Minimum | Maximum |
|---|---|---|
| Parameter | Value | Value |
| Distance from exit to site (cm) | 8.5 | 35 |
| Application (inhalation) breaths | 2 | 45 |
| Number of treatments | 1 | 42 |
| Length of time between treatments (hours) | 3 | 48 |
| Temperature of plasma stream at contact with | 10 | 40 |
| Site ° C. | | |
| Velocity of Plasma Stream at contact with Site | 0.5 | 200 |
| (m/sec) | | |

### Example 15

TABLE 15C
Pathogens

Streptococcus pyogenes a
Group A streptococcus in Streptococcal
pharyngitis (“Strep Throat”)
Haemophilus influenzae
Streptococcus pneumoniae
Corynebacterium diphtheriae
Bordetella pertussis
Bacillus anthracis
Streptococcus pneumonia
chlamydophila pneumoniae
mycoplasma pneumoniae'
staphylococcus aureus
moraxella catarrhalis
legionella pneumophila
mycobacterium tuberculosis
mycobacterium bovis
mycobacterium africanum
mycobacterium canetti
mycobacterium microti

### Example 15

TABLE 15D
Conditions

Flu
common cold
laryngitis
sinusitis
tonsillitis
bronchitis
pneumonia
bronchiolitis
tuberculosis
rhinitis
laryngotracheitis
tracheitis
epiglotitis
nasopharyngitis

### Example 16

### Treatment Scheme

### Viruses

This disclosed use of NO in this Example is for the use of NO to treat viral infections. An exemplary composition of matter in a plasma state for use in treating viruses is shown in Table 16A below. It will be appreciated that this composition itemization is merely exemplary, and that other compositions can also be used to beneficial effect. Minimum treatment values and maximum treatment values in Tables 16A and 16B are based on severity of infection. Severity of the infection is determined by the symptoms, surface area, depth, colony count. Application of therapy increases in intensity, duration and frequency as the severity increases. Minimum treatment parameters define the requirements for initiation of the decolonization process.

Exemplary pathogens are included in Table 16C, while an exemplary list of conditions that present with the pathogens of Table 16C are included in Table 16D.

### Example 16

TABLE 16A
PLASMA COMPOSITION

| | Minimum | | Maximum | |
|---|---|---|---|---|
| Parameter | Value | | Value | |
| Nitric Oxide (NO) | 200 | ppm | 800 | ppm |
| NO₂ | 0 | ppm | 25 | ppm |
| N₂ | 75 | vol % | 78 | vol % |
| O₂ | 18 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

### Example 16

TABLE 16B

| | Minimum | Maximum |
|---|---|---|
| Parameter | Value | Value |
| Distance from exit to site (cm) | 6 | 25 |
| Time of Application (sec/cm²) | 5 | 30 |
| Number of treatments | 1 | 20 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

### Example 16

TABLE 16C
Pathogens

Herpes Simplex
Herpes Zoster
herpes gladiatorum
viral warts
molluskum contagiosum
human papilloma virus
measles
rubella
erythema infectiosum
pityriasis rosea
echovirus
adenovirus
coxsakievirus

### Example 16

TABLE 16D
Conditions

Venous Ulcers
Pressure Ulcers
Decubetus Ulcers
Sickle Cell Ulcerations
Pyodermas
Ulerated Lesions
Vasculitis
Diabetic Foot Ulcers
Folliculitis
Cellulitis
Myocitis
Infections from Animal Bites
Sugical Site Infections
Catheterizations
Carbuncles
Furuncles
Abcesses
Erysipeloid
Erysipelas
Keratolysis
Dermatitis
Skin Tuberculosis
Impetigo
Actinomytosis
Leishmaniasis
Hepes Simplex
Herpetic Neuralgia
Skin Flaps
Skin Grafts
Burns
Traumatic Wounds
Complicated SSTI

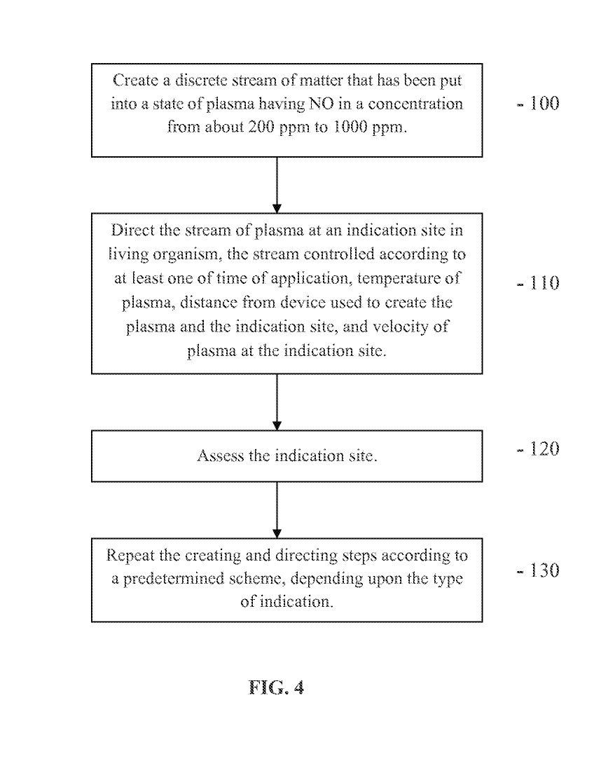

Referring now to FIG. 4, a flow diagram illustrating an exemplary method for administering NO in a plasma state to a treatment site in accordance with the present disclosure is shown. At a first step 100 of the exemplary method, a discrete stream of matter that has been put into a state of plasma may be created, in which the stream has, as part of its content, NO in a concentration from about 200 ppm to 1000 ppm. At step 110, the stream of matter in a plasma state is directed at an indication site in living organism, where the stream is controlled according to at least one of time of application, temperature of the matter in a plasma state, distance from device used to create the matter in a plasma state and the indication site, and velocity of matter in a plasma state at the indication site. At step 120, the indication site is assessed. At step 130, the creating and directing steps are repeated according to a predetermined scheme, depending upon the type of indication.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

While the present invention has been disclosed with reference to certain embodiments, numerous modifications, alterations and changes to the described embodiments are possible without departing from the sphere and scope of the present invention, as defined in the appended claim(s). Accordingly, it is intended that the present invention not be limited to the described embodiments, but that it has the full scope defined by the language of the following claims, and equivalents thereof.

1. A method for administering nitric oxide in a plasma state to an organism, the method comprising:
creating a discrete stream of matter that has been put into a state of plasma, in which the stream has, as part of its content, nitric oxide (NO) in a concentration from about 200 ppm to 1000 ppm; and
directing the discrete stream of matter at a site of action to obtain a therapeutic result by at least one of stimulating sub-dermal production of NO, mobilization of NO in a plasma state across and through the dermis, and the direct effect of NO in a plasma state on pathogens.

2. The method of claim 1, wherein directing the discrete stream of matter at a site of action comprises positioning a device used to create the discrete stream of matter from 1 to 25 centimeters from the device to the site.

3. The method of claim 1, wherein directing the discrete stream of matter at a site of action comprises directing the discrete stream of matter for 5 to 45 seconds for each square centimeter of site area.

4. The method of claim 1, wherein directing the discrete stream of matter at a site of action comprises inhalation of the discrete stream of matter for a predetermined number of breaths.

5. The method of claim 1, wherein the discrete stream of matter is directed to the site over a plurality of discrete application procedures, wherein the plurality of discrete application procedures is from 1 to 24 discrete applications.

6. The method of claim 1, further comprising directing the discrete stream of matter to the site over multiple discrete time periods, the time periods separated by from 3 to 168 hours.

7. The method of claim 1, wherein the temperature of the stream at contact with the site is from 10 to 60 degrees C.

8. The method of claim 1, wherein the velocity of the stream at contact with the site is from 0.5 to 200 meters per second.

9. The method of claim 1, wherein the stream of nitric oxide plasma is creating using an electric arc discharge device.

10. The method of claim 1, wherein the stream of nitric oxide plasma is creating using a microwave discharge device.

11. The method of claim 1, wherein the stream of nitric oxide plasma is creating using a gliding arc discharge device.

12. The method of claim 1, wherein the therapeutic result is selected from the list consisting of facilitating hair-growth, reducing wrinkles, facilitating vasodilation, alleviating pain associated with osteoarthritis and Rheumatoid Arthritis, combating Gram Positive microorganisms, combating Gram Negative microorganisms, combating fungi and viruses, combating asthma, combating respiratory infections, combating pre insult keloidosis, and combating post insult keloidosis.

13. The method of claim 1, wherein the therapeutic result is selected from the list consisting of treating osteoporosis, collagen formation, stem cell signaling, satellite cell differentiation, wound healing, wound management, acne, nerve regeneration, inhibiting cancer cell proliferation, promoting apoptosis, stimulating endogenous nitric oxide production, and stimulating iNOS pathways.

14. The method of claim 1, wherein the therapeutic result is selected from the list consisting of maintaining homeostasis in the cardiovascular and respiratory systems, causing vasodilation, easing blood pressure, cleaning the blood, reversing atherosclerosis, preventing cardiovascular diseases, slowing down atherosclerotic plaque deposition on vascular walls, suppressing bacterial infections, suppressing viruses and parasitic attacks, deterring cancer cell growth, reducing pain associated with joint swelling in arthritis, decreasing the risk of cancer, diabetes, myocardial infarction and stroke.

15. The method of claim 1, wherein the therapeutic result is selected from the list consisting of facilitating hair-growth, reducing wrinkles, facilitating vasodilation, alleviating pain associated with osteoarthritis and Rheumatoid Arthritis, combating Gram Positive microorganisms, combating Gram Negative microorganisms, combating fungi and viruses.

16. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 5 | 45 |
| Number of treatments | 1 | 24 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

17. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 10 | 90 |
| Number of treatments | 1 | 24 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

18. The method of claim 1, wherein directing the discrete stream of matter at a site of action is undertaken according to the following criteria:

| | | Maximum |
|---|---|---|
| Parameter | Minimum Value | Value |
| Distance from exit to site (cm) | 1 | 25 |
| Time of Application (sec/cm²) | 1 | 45 |
| Number of treatments | 1 | 200 |
| Length of time between treatments (hours) | 3 | 168 |
| Temperature of plasma stream at contact | 10 | 60 |
| with Site ° C. | | |
| Velocity of Plasma Stream at contact with | 0.5 | 200 |
| Site (m/sec) | | |

19. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 5 | 90 |
| Number of treatments | 1 | 200 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

20. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 5 | 45 |
| Number of treatments | 1 | 24 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

21. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 10 | 30 |
| Time of Application (sec/cm²) | 10 | 90 |
| Number of treatments | 1 | 200 |
| Length of time between treatments (hours) | 1 | 168 |
| Temperature of plasma stream at contact with | 10 | 50 |
| Site ° C. | | |
| Velocity of Plasma Stream at contact with Site | 0.5 | 200 |
| (m/sec) | | |

22. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 5 | 25 |
| Time of Application (sec/cm²) | 10 | 45 |
| Number of treatments | 3 | 40 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

23. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 15 | 120 |
| Number of treatments | 3 | 50 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

24. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 3 | 25 |
| Time of Application (sec/cm²) | 30 | 90 |
| Number of treatments | 3 | 40 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

25. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 1 | 25 |
| Time of Application (sec/cm²) | 20 | 120 |
| Number of treatments | 3 | 60 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

26. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 6 | 25 |
| Time of Application (sec/cm²) | 10 | 30 |
| Number of treatments | 10 | 100 |
| Length of time between treatments (hours) | 12 | 72 |
| Temperature of plasma stream at contact with | 10 | 60 |
| Site ° C. | | |
| Velocity of Plasma Stream at contact with Site | 0.5 | 200 |
| (m/sec) | | |

27. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 6 | 25 |
| Time of Application (sec/cm²) | 5 | 30 |
| Number of treatments | 4 | 24 |
| Length of time between treatments (hours) | 12 | 168 |
| Temperature of plasma stream at contact with | 10 | 60 |
| Site ° C. | | |
| Velocity of Plasma Stream at contact with Site | 0.5 | 200 |
| (m/sec) | | |

28. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 6 | 25 |
| Time of Application (sec/cm²) | 10 | 30 |
| Number of treatments | 1 | 12 |
| Length of time between treatments (hours) | 3 | 72 |
| Temperature of plasma stream at contact with | 10 | 60 |
| Site ° C. | | |
| Velocity of Plasma Stream at contact with Site | 0.5 | 200 |
| (m/sec) | | |

29. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 8.5 | 35 |
| Application (inhalation) breaths | 2 | 45 |
| Number of treatments | 1 | 42 |
| Length of time between treatments (hours) | 3 | 48 |
| Temperature of plasma stream at contact with | 10 | 40 |
| Site ° C. | | |
| Velocity of Plasma Stream at contact with Site | 0.5 | 200 |
| (m/sec) | | |

30. The method of claim 1, wherein directing the discrete stream of matter at a site of action is undertaken according to the following criteria:

| | Minimum | Maximum |
|---|---|---|
| Parameter | Value | Value |
| Distance from exit to site (cm) | 6 | 25 |
| Time of Application (sec/cm²) | 5 | 30 |
| Number of treatments | 1 | 20 |
| Length of time between treatments (hours) | 3 | 168 |
| Temperature of plasma stream at contact with | 10 | 60 |
| Site ° C. | | |
| Velocity of Plasma Stream at contact with Site | 0.5 | 200 |
| (m/sec) | | |

31. The method of claim 1, wherein the discrete stream of matter comprises:

| | Minimum | | Maximum | |
|---|---|---|---|---|
| Parameter | Value | | Value | |
| Nitric Oxide (NO) | 200 | ppm | 1000 | ppm |
| NO₂ | 0 | ppm | 25 | ppm |
| N₂ | 75 | vol % | 78 | vol % |
| O₂ | 18 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 300 | ppm | 500 | ppm |
| H2 | 0 | ppm | 10000 | ppm |

32. The method of claim 1, wherein the discrete stream of matter comprises:

| | Minimum | | Maximum | |
|---|---|---|---|---|
| Parameter | Value | | Value | |
| Nitric Oxide (NO) | 50 | ppm | 800 | ppm |
| NO₂ | 0 | ppm | 25 | ppm |
| N₂ | 75 | vol % | 95 | vol % |
| O₂ | 3 | vol % | 21 | vol % |
| O₃ | 0 | ppm | 0.1 | ppm |
| H₂O₂ | 0 | ppm | 1500 | ppm |
| H₂O | 0 | ppm | 20000 | ppm |
| Ar | 1 * 10\hat{ }4 | ppm | 9.1 * 10\hat{ }5 | ppm |
| He | 5.2 | ppm | 9.1 * 10\hat{ }5 | ppm |
| CO | 0 | ppm | 50 | ppm |
| CO₂ | 0 | ppm | 500 | ppm |
| H2 | 0 | ppm | 10000 | ppm |
